(19) United States
(12) Patent Application Publication
(10) Pub. No.: US 2024/0424301 A1
(43) Pub. Date: Dec. 26, 2024

(54) NEUROLOGICAL TREATMENT SYSTEM

(71) Applicant: Covidien LP, Mansfield, MA (US)

(72) Inventor: Alan Eskuri, Irvine, CA (US)

(21) Appl. No.: 18/825,005

(22) Filed: Sep. 5, 2024

Related U.S. Application Data
(63) Continuation of application No. 14/019,469, filed on Sep. 5, 2013.
(60) Provisional application No. 61/697,432, filed on Sep. 6, 2012.
(60) Provisional application No. 61/739,977, filed on Dec. 20, 2012.

(51) Int. Cl. A61N 1/36; A61B 17/22; A61N 1/05

(52) U.S. Cl. CPC A61N 1/36082; A61B 17/22; A61N 1/0529; A61N 1/0551; A61N 1/36185

(57) ABSTRACT

A neurological treatment apparatus includes a first endovascular device comprising a plurality of first electrodes extending along a first length in a distal portion of the first device, the distal portion of the first device being configured for endoluminal navigation into cerebral vasculature, and a second endovascular device comprising a plurality of second electrodes extending along a second length in a distal portion of the second device, configured for endoluminal navigation into the cerebral vasculature, such that electrostimulative current may be passed between the first and second electrodes.

### RELATED APPLICATIONS

The present application is a continuation of U.S. patent application Ser. No. 14/019,469, filed on Sep. 5, 2013, which claims the benefit of each of U.S. Pat. App. Ser. No. 61/697,432, filed on Sep. 6, 2012, and U.S. Pat. App. Ser. No. 61/739,977, filed on Dec. 20, 2012. The entire contents of each of the above referenced applications is incorporated by reference, as if fully set forth herein.

### FIELD

The present disclosure relates to neurological treatment. In particular, the present disclosure relates to neurological treatment by electrostimulation along pathways within brain tissue.

### BACKGROUND

Neurodegenerative diseases and their effects can include Alzheimer's Disease, Parkinson's disease, Huntington's disease, tremor, epilepsy, and/or ischemia of the brain, such as stroke. These may include or lead to progressive loss of structure or function of neurons, including death of neurons.

### SUMMARY

The subject technology is illustrated, for example, according to the following non-limiting summary of some embodiments disclosed herein. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 55. The other clauses can be presented in a similar manner.

Clause 1. A method, comprising:
inserting a first endovascular device within a first cerebral blood vessel, the first endovascular device comprising a plurality of first electrodes extending along a first length in a distal portion of the first device;
inserting a second endovascular device within a second cerebral blood vessel, the second endovascular device comprising plurality of second electrodes extending along a second length in a distal portion of the second device;
passing electrical currents along a plurality of pathways between the first electrodes and the second electrodes, wherein at least a portion of the pathways pass through at least one of the hippocampus or dentate gyrus.

Clause 2. The method of clause 1, wherein inserting the second endovascular device comprises placing the second electrodes on a side of the hippocampus or dentate gyrus opposite the first electrodes.

Clause 3. The method of clause 1, wherein passing electrical currents comprises passing said currents between a plurality of said first electrodes and a plurality of said second electrodes.

Clause 4. The method of clause 1, wherein said currents comprise neurostimulative currents.

Clause 5. An apparatus comprising:
a first endovascular device comprising a plurality of first electrodes extending along a first length in a distal portion of the first device, the distal portion of the first device being configured for endoluminal navigation into cerebral vasculature;
a second endovascular device comprising a plurality of second electrodes extending along a second length in a distal portion of the second device, the distal portion of the second device being configured for endoluminal navigation into cerebral vasculature;
a power source in electrical communication with the first electrodes and the second electrodes;
a controller in communication with the power source; and
a machine-readable medium comprising instructions executable by the controller to operate the power source so as to establish a network of neurostimulation current pathways between the first electrodes and the second electrodes.

Clause 6. The apparatus of clause 5, wherein:
one of said endovascular devices comprises a proximally located electrode;
the other of said endovascular devices comprises a distally located electrode; and
said network includes at least one neurostimulation current pathway between said proximally located electrode and said distally located electrode.

Clause 7. The apparatus of clause 5, wherein said network comprises first neurostimulation pathways that extend mainly or only laterally, and second neurostimulation pathways that extend both laterally and longitudinally.

Clause 8. The apparatus of clause 5, wherein said algorithm is executable by the controller to operate the power source so as to pass neurostimulative electrical current along said current pathways.

Clause 9. The apparatus of clause 8, wherein said electrical current is configured to regenerate neurocytes in the tissue through which said network passes.

Clause 10. An apparatus comprising:
a first endovascular device comprising a plurality of first electrodes extending along a first length in a distal portion of the first device, the distal portion of the first device being configured for endoluminal navigation into cerebral vasculature;
a second endovascular device comprising a plurality of second electrodes extending along a second length in a distal portion of the second device, the distal portion of the second device being configured for endoluminal navigation into the cerebral vasculature; and
means for passing electrostimulative current between the first electrodes and the second electrodes.

Clause 11. The apparatus of clause 10, wherein said means further comprises means for establishing a network of neurostimulation current pathways between the first electrodes and the second electrodes.

Clause 12. The apparatus of clause 11, wherein said network comprises first neurostimulation pathways that extend mainly or only laterally, and second neurostimulation pathways that extend both laterally and longitudinally.

Clause 13. The apparatus of clause 10, wherein said means comprises a controller and program instructions accessible by said controller.

Clause 14. The apparatus of clause 10, wherein said means is configured for at least one of:
regenerating cerebral neurocytes;
inhibiting or reversing the formation of neurofibrillary tangles;
inhibiting or reversing the formation of amyloid plaques;
inhibiting or reversing the attachment of amyloid plaques to neurocytes;
inhibiting the cellular apoptosis cascade associated with ischemic stroke; or increasing rate and volume of pharmacologic agent delivery.

Clause 15. A method, comprising:
providing a first treatment to increase blood flow downstream of an obstruction at an obstruction location within a blood vessel of a patient;
inserting a first endovascular device downstream of the obstruction location, the first endovascular device comprising a plurality of first electrodes extending along a first length in a distal portion of the first device;
inserting a second endovascular device downstream of the obstruction location, the second endovascular device comprising plurality of second electrodes extending along a second length in a distal portion of the second device;
passing electrical currents along a plurality of pathways between the first electrodes and the second electrodes, wherein at least a portion of the pathways pass through a region of brain tissue affected by the obstruction.

Clause 16. The method of clause 15, wherein the first treatment comprises mechanically removing, lysine, breaking up, or aspirating the obstruction.

Clause 17. The method of clause 15, wherein the first treatment comprises administering a blood thinner to the patient.

Clause 18. The method of clause 15, wherein inserting the first endovascular device comprises inserting the first endovascular device into a first blood vessel downstream of the obstruction location.

Clause 19. The method of clause 18, wherein inserting the second endovascular device comprises inserting the second endovascular device into a second blood vessel, different from the first blood vessel, downstream of the obstruction location.

Clause 20. The method of clause 15, wherein the region of brain tissue affected by the obstruction comprises an infarction.

Additional features and advantages of the subject technology will be set forth in the description below, and in part will be apparent from the description, or may be learned by practice of the subject technology. The advantages of the subject technology will be realized and attained by the structure particularly pointed out in the written description and claims hereof as well as the appended drawings.

It is to be understood that both the foregoing general description and the following detailed description are exemplary and explanatory and are intended to provide further explanation of the subject technology as claimed.

### BRIEF DESCRIPTION OF THE DRAWINGS

The accompanying drawings, which are included to provide further understanding of the subject technology and are incorporated in and constitute a part of this specification, illustrate aspects of the subject technology and together with the description serve to explain the principles of the subject technology.

FIG. 1 is a schematic view of a vasculature of a brain.

FIG. 2 is a schematic view of a neurological treatment system.

FIG. 3A is a schematic view of the neurological treatment system of FIG. 2 and a method of using the system to treat brain tissue.

FIG. 3B is a schematic view of a neurological treatment system and a method of using the system to treat brain tissue.

FIG. 4A is a schematic view of an obstructed blood vessel and a stage of a method of using the neurological treatment system of FIG. 2 to treat brain tissue.

FIG. 4B is a schematic view of a mechanical thrombectomy device and a stage of a method of using the neurological treatment system of FIG. 2 to treat brain tissue.

FIG. 4C is a schematic view of the neurological treatment system of FIG. 2 and a stage of a method of using the system to treat brain tissue.

FIG. 4D is a schematic view of the neurological treatment system of FIG. 2 and a stage of a method of using the system to treat brain tissue.

### DETAILED DESCRIPTION

In the following detailed description, numerous specific details are set forth to provide a full understanding of the subject technology. However, the subject technology may be practiced without some of these specific details. In other instances, well-known structures and techniques have not been shown in detail so as not to obscure the subject technology.

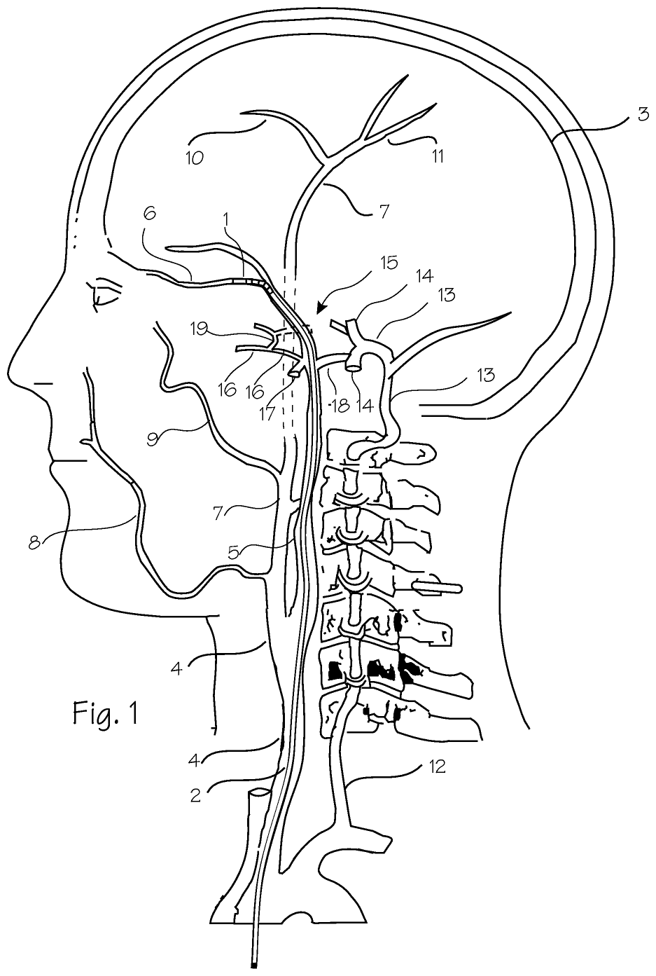

FIG. 1 shows the vasculature of the brain. The brain 3 is supplied with blood through the carotid and the vertebral arteries on each side of the neck. The arteries include the common carotid artery 4 in the neck, which is a common access pathway for the various devices and/or methods disclosed herein, the internal carotid 5 which supplies the ophthalmic artery 6. The external carotid 7 supplies the maxillary artery 8, the middle meningeal artery 9, and the superficial temporal arteries 10 (frontal) and 11 (parietal). The vertebral artery 12 supplies the basilar artery 13 and the cerebral arteries including the posterior cerebral artery 14 and the circle of Willis indicated generally at 15. The siphon of the vertebral artery appears in the intra-cranial vasculature on the vertebral approach to the Circle of Willis. Also supplied by the internal carotid artery are the anterior cerebral artery 16 and the middle cerebral artery 17, as well as the circle of Willis, including the posterior communicating artery 18 and the anterior communicating artery 19. The siphon of the internal carotid artery 5 appears in the intra-cranial vasculature on the carotid approach into the Circle of Willis. These arteries typically have an internal diameter of about 1 mm to 5 mm, most commonly from 2-4 mm. The methods and devices described herein allow access to these arteries for treatment(s). In FIG. 1, an insertion catheter 2 (which can comprise, for example, a microcatheter) is shown extending through the common carotid artery 4 and the internal carotid artery 5, with a device 1 extending through the catheter 2 and into the anterior cerebral artery 16.

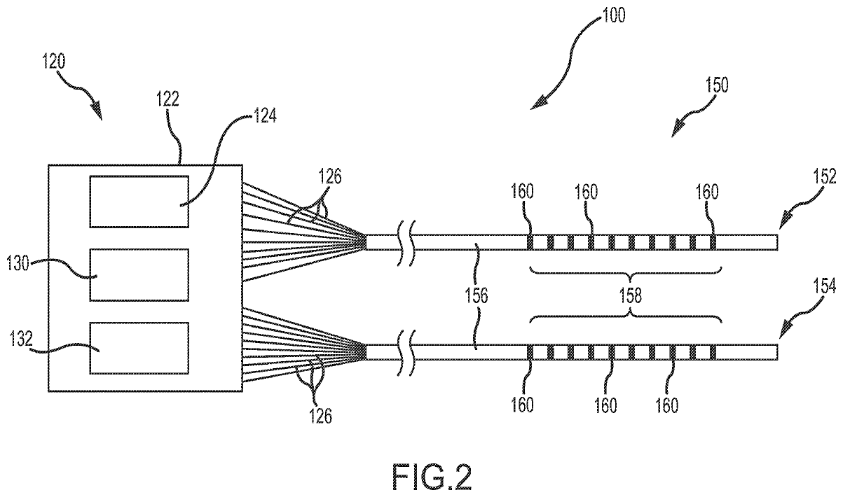

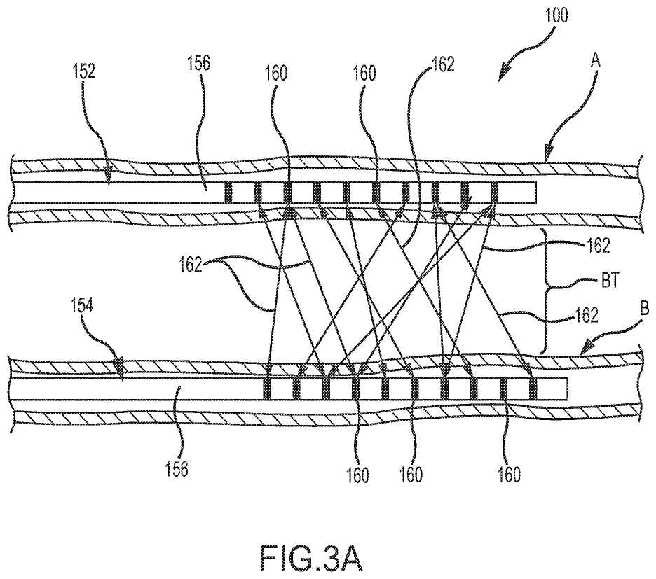

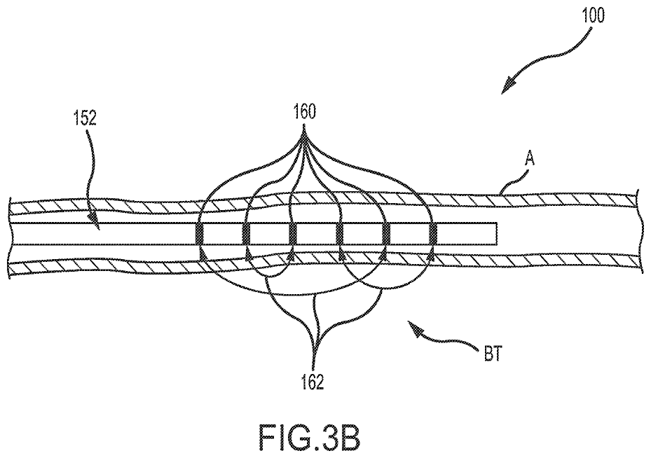

FIGS. 2 and 3A-3B depict a neurological treatment system 100 that can be used to treat neurological disorders such as Alzheimer's disease by applying energy to the affected tissue, such as brain tissue and/or other neurological tissues. The system 100 can comprise an energy generation portion 120 that is coupled or configured to be coupled to an energy application portion 150, so that energy developed by the generation portion 120 can be transmitted to, and applied to tissue by, the application portion 150.

In one embodiment, the energy generation portion 120 can comprise an electrical generator 122 configured to output medically useful electrical current, which can be electrostimulative, electrosurgical and/or ablative current. The generation portion 120 can include a suitable controller 124 that can be used to control various parameters of the energy output by the portion 120/generator 122, such as intensity, amplitude, duration, frequency, duty cycle, polarity, etc. The energy application portion 150 can comprise one or more endovascular devices (such as the depicted pair of first and second endovascular devices 152, 154), or an endovascular device and an extracorporeal conductive device such as a grounding pad.

One or both of the endovascular devices 152, 154 can comprise an elongate, flexible shaft 156 that is sized and configured for navigation through blood vessels such as cerebral blood vessels. The shaft 156 can be a tube or other elongate member formed from a flexible, preferably electrically insulative material. Suitable materials for the shaft 156 include medically acceptable polymers, or metals (e.g. in the form of a hypotube) coated with a suitable electrical insulator, or combinations thereof. The endovascular devices 152, 154 can each further comprise an energy application region 158 which can in turn comprise one or more (e.g., 1, 2, 3, 4, 5, 8, 10, 12, 15, or 20 or more) electrodes 160. The devices 152, 154 can each have the same number of electrodes, or the device 152 can have a number of electrodes 160 that is different from the number of electrodes of the device 154.

The electrodes 160 can comprise metallic or other conductive rings, or coils or other suitable conductive structures, and can each form an outer conductive surface that is exposed and configured for electrically conductive contact with adjacent tissue, such as the inner wall of a blood vessel, and/or brain tissue or other neurological tissue being treated by the system 100. Some or all of the outer conductive surfaces can extend partially or completely circumferentially around the longitudinal axis of the endovascular device 152/154. The outer conductive surfaces can be approximately flush with the outer surface of the shaft 156, or they can extend radially outward from the outer surface of the shaft 156, e.g. in a bulged or toroidal shape. The electrodes 160 can have a fixed outer diameter or size, or a radially expandable outer diameter or size. One, some, or all of the electrodes 160 can be “painted” electrodes.

During use, one or both of the endovascular devices 152, 154 can be employed to apply energy to the tissue being treated. For example, FIG. 3A schematically depicts the use of both endovascular devices 152, 154 to treat brain tissue BT, which can be located within a cranium. To facilitate treatment of the brain tissue BT, the devices 152, 154 can be inserted into or otherwise located in blood vessels A, B, which can be cerebral, intracranial, pericranial, and/or neurovascular blood vessels. Alternatively, one or both of the devices 152, 154 can be inserted or implanted directly into the brain tissue BT.

After insertion, one or both of the devices 152, 154 can be energized to apply energy to the brain tissue BT. For example, one or more, or all, of the electrodes 160 of the first endovascular device 152 can be energized and pass electrical current to one or more, or all, of the electrodes 160 of the second endovascular device 154. This can be accomplished by activating a current source (e.g. the generator 122) connected to the energized electrode(s) of the first endovascular device 152 by leads 126, or activating those portions of the current source 122 that are connected to the energized electrode(s), and/or at least temporarily (e.g. electronically via the controller 124) switching the energized electrode(s) into electrical communication with the current source 122 or the activated portion(s) thereof. While the electrode(s) 160 of the first device 152 are energized in this manner to thereby serve as “active” electrode(s), one or more of the electrodes 160 of the second device 154 can be connected to ground and thereby serve and ground electrodes that receive current propagating from the active electrode(s). This can be accomplished by at least temporarily (e.g. electronically via the controller 124) switching the selected ground electrode(s) into electrical communication with ground, or simply leaving the selected ground electrodes in electrical communication with ground throughout the desired treatment period. One or more electrode pairs can thereby be formed between at least one active electrode and at least one ground electrode, and electrical current 162 passed across the electrode pair from the active to the ground electrode.

During operation, in one embodiment, each electrode 160 of both endovascular devices 152, 154 can be bifunctional; that is, each electrode 160 can serve as either an active electrode or a ground electrode at different points in time as the treatment proceeds. Electrode pairs can be established temporarily and the constituent electrodes selected randomly or in a preset pattern to form a large variety of conduction pathways or currents 162 during the treatment cycle. For example, an electrode pair can be formed temporarily from a relatively distally located electrode 160 of the second device 154 (serving, e.g., as the active electrode) and a relatively proximally located electrode 160 of the first device 152 (serving, e.g., as the ground electrode), thereby creating a pathway or current 162 that traverses both longitudinally and laterally through the brain tissue BT, from the second device 154 to the first device 152. Instead of or in addition to currents/pathways of this type, electrode pairs can be formed from electrodes that are longitudinally closer together, so that the resulting current traverses mainly or only laterally through the brain tissue BT. If desired, during the period of time that a given electrode pair is formed, the polarity can be switched once or repeatedly, to create currents traveling in either direction along the pathway.

Preferably, during operation the system 100 uses all (or a large proportion) of the available current pathways at various times during the treatment cycle. Thus is formed a relatively dense “stimulation matrix” or network as depicted by the current pathways in FIG. 3A, and a relatively large proportion of the volume of the treated tissue BT is located on or near a conduction pathway 162, as the system 100 sequentially or simultaneously activates a large selection of available electrode pairs during the treatment period. For example, a first pair of electrodes 160 may form a pathway 162 in a first operation. Subsequently, a second pair of electrodes 160, different from the first pair of electrodes 160, may form a different pathway 162 in a second operation. Alternatively or in combination, a first pair of electrodes 160 and the second pair of electrodes 160 may form respective pathways 162 simultaneously.

The controller 124 or other suitable hardware can execute an algorithm or program instructions, stored in memory accessible by the controller 124, to activate electrode pairs or sets in a sequence that is preset, random or otherwise. The controller 124 may provide a module for executing such an algorithm or program instructions. Further provided are a processor 130 for executing instructions and a machine-readable medium 132, such as a volatile or non-volatile memory, for storing data and/or instructions. The instructions, which may be stored in a machine-readable medium, may be executed by the controller 124.

Where the first and second device 152, 154 contain X and Y electrodes, respectively, the devices collectively form 2XY possible conduction pathways (comprising only one electrode from the first device and only one electrode from the second device), taking into account that each conduction pathway can operate in two directions or polarities. Preferably, the algorithm or program instructions call for use of all, or significantly more than half, of the 2XY available pathways during a single treatment period with the system 100. In various embodiments, X and Y can each be greater than or equal to 5, or greater than or equal to 10, or any other suitable number or range disclosed herein.

As shown in FIG. 3B, instead of or in addition to the “two-device” pathways depicted in FIG. 3A, “single-device” pathways may be formed and employed, using two or more electrodes 160 on a single device 152 to form an electrode pair or set and pathway 162 through brain tissue BT. In any operation of the system 100, one or both devices 152, 154 can be moved or reciprocated longitudinally within the treated tissue (e.g. brain tissue BT) to move the resulting conduction pathways within the tissue, increasing the proportion of treated tissue.

The current 162 applied by the system 100 can be any suitable therapeutic current. For example, the current 162 can be electrostimulative current, and the generator 122 can comprise an electrostimulation generator. Where the treated tissue comprises the brain tissue BT, any electrostimulation current suitable for brain tissue can be employed. Other suitable therapeutic current includes radiofrequency current, or any therapeutic or ablative alternating or direct current. Instead of or in addition to the electrodes 160, suitable antennae may be employed on the device(s) 152, 154 to apply microwave energy to the treated tissue. Alternatively, the device(s) 152, 154 can be configured to direct light energy (e.g. infrared laser) into or through the desired treated tissue. Multiple such emitters, for example, fiber optics, can be arranged in an array similar to the electrode arrays shown in FIGS. 2 and 3A-3B. The laser wavelength can be selected to either propagate through or be absorbed by the treated tissue, to a desired degree in either case.

When the system 100 is employed to treat Alzheimer's disease, the treated brain tissue BT can comprise the hippocampus and/or dentate gyrus, and the devices 152, 154 (e.g., the electrodes 160 thereof, or other energy emitters) can be inserted into vascular locations near either such structure, e.g. in vascular locations on opposite sides of either structure. For example, the devices 152, 154 can be inserted into the cerebral arteries (i.e., Posterior, Middle, Anterior and/or Basilar), and then employed to apply energy, such as electrostimulative current as described above and/or depicted in FIGS. 3A-3B to the hippocampus and/or dentate gyrus. Thus, the applied electrostimulative current can take the form of neurostimulative current, including any such current that is suitable for regenerating cerebral neurocytes. The applied current can therefore treat Alzheimer's disease by regenerating cerebral neurocytes, and/or inhibiting or reversing the formation of neurofibrillary tangles or amyloid plaques, in the treated area. In one embodiment, a method comprises administering to a patient electrostimulative or neurostimulative current using any apparatus or technique disclosed herein.

The device(s) 152, 154 can be delivered to and/or through any one or more of a number of vessels to access a treatment region. One or more blood vessels may be utilized to reach the target region. The device(s) 152, 154 may span, straddle, or encompass a treatment region based on location within one or more blood vessels.

The device(s) 152, 154 can stimulate the cortex of the brain or the deep brain to provide post-stroke rehabilitation (from hemorrhagic stroke, ischemic stroke or head/brain trauma), Parkinson's disease, essential tremor, Huntington's disease, Alzheimer's disease, epilepsy, depression, obsessive compulsive disorder, schizophrenia, and neuropathic pain. Any lobe of the cortex or deep brain can be stimulated, for example, the cortical region of the brain, the motor strip, sensor strip, and/or premotor cortex, inter alia. Examples of arteries providing access to the cortex include any of the branches off of the external carotid, maxillary, or meningeal arteries. Examples of veins providing access to the cortex include the superior sagittal sinus, any of the superior cerebral veins branching from the superior sagittal sinus (e.g., the lacuna, frontopolar vein, anterior frontal vein, posterior frontal vein, precentral vein, central vein, anterior parietal vein, posterior parietal vein, and occipital vein), superior sylvian vein, vein of Labbe, vein of Trolard, inferior sagittal sinus, and any inferior cerebral veins branching off of the inferior sagittal sinus, transverse sinus, and meningeal sinus.

The device(s) 152, 154 can stimulate the deep brain region by accessing the anterior thalamus, ventrolateral thalamus (Thal), internal segment of globus pallidus (GPi), substantia nigra pars reticulata (SNr), subthalamic nucleus (STN), external segment of globus pallidus (GPe), neostriatum, cingulate, and cingulate gyrus, inter alia. Examples of arteries providing access to the deep brain include any branches off of the internal carotid or vertebral arteries. Examples of veins providing access to the deep brain include the inferior sagittal sinus, pericallosal sinus, cavernous sinus, sphenoid sinus, temperal basal vein, and occipital veins.

The device(s) 152, 154 can stimulate the spheno palatine ganglion (SPG), which can control the amount of blood flow to the brain and the permeability of the blood brain barrier, e.g., to hyperperfuse a hemisphere of the brain damaged as a result of an ischemic event, such as a stroke, or to help metabolize amlyoid plaques caused by Alzheimer's Disease and prevent the occurrence of vaso-spasms, both achieved through increased blood flow to the brain. Examples of arteries providing access to the SPG include the maxillary artery, descending palatine artery, and facial artery. Examples of veins providing access to the SPG include the superficial temporal veins and the facial vein.

The various embodiments of the system 100 disclosed herein can be employed in still further applications. For example, any of the disclosed embodiments of the system 100 can be employed to promote local uptake or tissue diffusion of vascularly delivered pharmacologic or biologic agents. To accomplish this, the system 100 (particularly the electrodes 160 of the device(s) 152, 154) can be employed to establish or maintain an electrical field in the presence of a pharmacologic or biologic agent, and thereby enhance the uptake and/or tissue diffusion of the agent(s). This can be performed in brain tissue BT as described in connection with FIGS. 3A-3B, or in other types of tissue. The electrical field established or maintained with the system 100 can be of an intensity that induces electro-permeation of the blood-brain barrier, e.g. at an intensity below that which induces electroporation. According to the above-disclosed methods, the various embodiments of the system 100 can be employed to enhance drug delivery through electropermeation of barrier tight junctions.

The various disclosed embodiments of the system 100 can also be used to accelerate neural stem cell differentiation and/or activation, e.g. into a functional network. The system 100 can be activated to electrically stimulate undifferentiated stem cells and induce potentiation to neurons. The system 100 can also be employed to electrically stimulate implanted pluripotent stem cells or previously cultured neurons, and cause them to populate and connect into functioning neural networks faster or more efficaciously. When used for the purposes described in this paragraph, the system 100 can be deployed, e.g. as described in connection with FIGS. 3A-3B, to provide endovascular or transvascular electrical stimulation to stem cells, cultured neurons or a combination of the two. In this manner the system can provide neurological therapy by enhancing or accelerating the formation or repair of neural networks.

The disclosed embodiments of the system 100 can also be used to heal, enhance or repair neural tissue by accelerating myelin sheath formation. For example, the system 100 can be deployed endovascularly into neural tissue, e.g. as described in connection with FIGS. 3A-3B, and be activated to electrically stimulate myelin sheath repair or formation in the targeted neural tissue. This may be done in a patient suffering from degenerated or ungenerated myelin sheaths.

The disclosed embodiments of the system 100 can also be used to inhibit the apoptosis cascade associated with ischemic stroke. For example, the system 100 can be deployed endovascularly into neural tissue, e.g. as described in connection with FIGS. 3A-3B, and be activated to electrically stimulate infarcted areas of targeted tissue. In this manner, the system 100 can inhibit the amount of neurological decline associated with ischemic stroke.

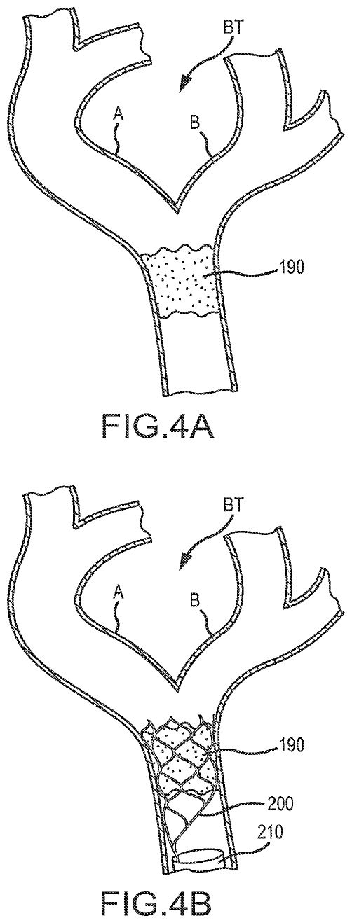

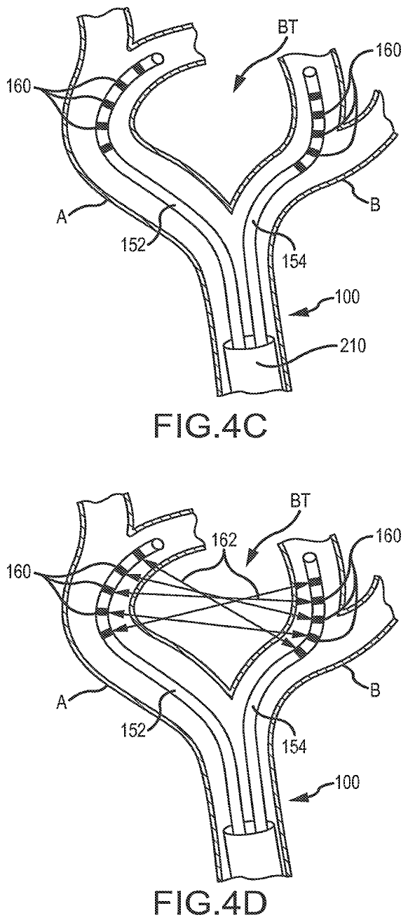

With reference to FIGS. 4A-4D, the disclosed embodiments of the system 100 can be used as a secondary treatment in conjunction with a primary treatment, such as mechanical thrombectomy and/or blood thinners. A primary treatment may be adapted to address conditions including deep vein thrombosis, pulmonary embolism, myocardial infarction, and stroke. For example, a stroke may be treated by restoration of blood flow and/or by removal of a thrombus or other obstruction in a blood vessel. Mechanical thrombectomy devices include any device capable of removing, lysing, breaking up, and/or aspirating a thrombus or other obstruction. Mechanical thrombectomy devices, such as stentrievers, may be employed to address a cause of an ischemic stroke. Examples of some mechanical thrombectomy devices and methods are disclosed in U.S. Pub. No. 2011/0060212, published on Mar. 10, 2011, and U.S. Pub. No. 2012/0083868, published on Apr. 5, 2012, and U.S. Pub. No. 2012/0316600, published on Dec. 13, 2012, the entire contents of each of which are incorporated herein by reference. Alternatively or in combination with the above, an aspiration device may be provided to remove a thrombus or other obstruction from a blood vessel by aspiration. Alternatively or in combination with the above, a drug therapy may be applied as a primary stroke treatment. For example, blood thinners (e.g., anticoagulants) may be provided to treat an ischemic stroke or enhance performance of a mechanical thrombectomy device. A primary treatment may be applied to address an obstruction at or near an internal carotid artery (ICA), a middle cerebral artery (MCA), an M1 bifurcation, a vertebral artery, a basilar artery or bifurcation, or other location in a vasculature.

As shown in FIG. 4A, an obstruction 190 partially blocks blood flow to each of the blood vessels A, B. The brain tissue BT near or between the blood vessels A, B may be affected by the obstruction 190. As shown in FIG. 4B, an exemplary mechanical thrombectomy device 200 (such as a stentriever and/or any other suitable mechanical thrombectomy device(s)) may be deployed from a catheter 210 to treat the obstruction 190. The obstruction 190 is removed, lysed, broken up, and/or aspirated.

As shown in FIG. 4C, a secondary treatment employing the disclosed embodiments of the system 100 may be applied, and can comprise operating the system 100 in any manner or method disclosed herein. For example, the devices 152, 154 may be navigated from the catheter 210 or another catheter through the blood vessels A, B so as to encompass an affected region of the brain tissue BT. The blood vessels A, B may be downstream of the location of the obstruction 190. The affected region of the brain tissue BT may have suffered an infarction, acute hypoxia, or be otherwise affected by inadequate blood flow during the stroke. As shown in FIG. 4D, one or more pathways or currents 162 are provided in the brain tissue BT between the electrodes 160 of the device 152 and the electrodes 160 of the device 154. While two devices 152, 154 are shown, the use of only one device 152 is also contemplated according to embodiments disclosed herein.

Operation of the device(s) 152, 154 provides stimulation to the affected region. For example, based on the location of an obstruction, a target region downstream of the location of the obstruction is identified. The target region of the brain tissue BT may be one or more of the brainstem, the cerebral cortex, the cerebellum, the parietal lobe, the temporal lobe, the frontal lobe, the spinothalamic tract, corticospinal tract, the dorsal column, the motor cortex, the sensory cortex, Wernicke's area, Broca's area, or any other region affected by a stroke. One or both of the device(s) 152, 154 are navigated vascularly to at least partially encompass the target region affected by the stroke. For example, one or both of the device(s) 152, 154 may be navigated to be near or encompass an affected region at, near, or downstream of an internal carotid artery (ICA), a middle cerebral artery, an M1 bifurcation, a vertebral artery, or a basilar artery or bifurcation. One or both of the device(s) 152, 154 may be navigated through the blood vessel in which the obstruction occurred. One or both of the device(s) 152, 154 provides stimulation to one or more affected regions to inhibit an amount of neurological decline associated with the stroke.

A secondary treatment employing the disclosed embodiments of the system 100 may be applied prior to, during, and/or after a primary treatment. A secondary treatment may be applied during the same procedure in which a primary treatment is employed. For example, the device(s) 152, 154 may provide a stimulation treatment for a period of time following reperfusion of a blood vessel. The stimulation may promote growth factors, thereby stimulating cellular growth. Applying a stimulation treatment immediately or otherwise after reperfusion can decrease the severity of a stroke, thereby reducing complications and cost associated with further recovery. Alternatively or in combination with the above, a secondary treatment employing the disclosed embodiments of the system 100 may be applied in a (second, third, fourth, etc.) procedure apart from the (first) procedure in which a primary treatment is employed. For example, the second, third, fourth and/or any other such subsequent procedure can be performed on a different day, and/or in a different week, month, or year from the first procedure.

While a secondary treatment may supplement a primary treatment for stroke, the disclosed embodiments of the system 100 can be used with other primary treatments to address conditions including deep vein thrombosis, pulmonary embolism, and myocardial infarction. In each case, a secondary treatment employing the disclosed embodiments of the system 100 may be performed prior to, during, or after a primary treatment of the condition (e.g., deep vein thrombosis, pulmonary embolism, and myocardial infarction). A target region affected by the condition is identified and stimulated.

A primary treatment may be applied to address other conditions, such as atherosclerosis, vasoconstriction, artery dissection, vasculopathy, Moyamoya disease, fibromuscular dysplasia, arterial embolus, and intracerebral hemorrhage. Corresponding primary treatments may be provided in conjunction with a secondary treatment employing the disclosed embodiments of the system 100.

The disclosed embodiments of the system 100 can be used to treat the effects of traumatic brain injury. Rapid acceleration or deceleration of the head can result in the corpus callosum shearing against the falx cerebri. The stimulation provided by the system 100 may promote growth factors, thereby stimulating cellular growth. Applying a stimulation treatment after traumatic brain injury may reduce the severity of its effects.

An exemplary treatment of the present disclosure has a duration of approximately one hour, or less than approximately one hour. For example, a treatment may include applying stimulation for about 10, 20, 30, 40, 50, 60, 70, 80, 90, 100, 110, or 120 minutes. A treatment session may be repeated multiple times with consistent or varied frequency within a given time period. For example, a treatment may be applied one or more times in a day, one or more times in a week, one or more times in a month, or one or more times in a year. An interval between consecutive treatment sessions may be one or more days, one or more weeks, one or more months, or one or more years.

As used herein, the word “module” refers to logic embodied in hardware or firmware, or to a collection of software instructions, possibly having entry and exit points, written in a programming language, such as, for example C++. A software module may be compiled and linked into an executable program, installed in a dynamic link library, or may be written in an interpretive language such as BASIC. It will be appreciated that software modules may be callable from other modules or from themselves, and/or may be invoked in response to detected events or interrupts. Software instructions may be embedded in firmware, such as an EPROM or EEPROM. It will be further appreciated that hardware modules may be comprised of connected logic units, such as gates and flip-flops, and/or may be comprised of programmable units, such as programmable gate arrays or processors. The modules described herein are preferably implemented as software modules, but may be represented in hardware or firmware.

In general, it will be appreciated that the processors can include, by way of example, computers, program logic, or other substrate configurations representing data and instructions, which operate as described herein. In other embodiments, the processors can include controller circuitry, processor circuitry, processors, general purpose single-chip or multi-chip microprocessors, digital signal processors, embedded microprocessors, microcontrollers and the like.

According to one aspect of the disclosure, a machine-readable medium is a computer-readable medium encoded or stored with instructions and is a computing element, which defines structural and functional interrelationships between the instructions and the rest of the system, which permit the instructions' functionality to be realized. In one aspect, a machine-readable medium is a non-transitory machine-readable medium, a machine-readable storage medium, or a non-transitory machine-readable storage medium.

Furthermore, it will be appreciated that in one embodiment, the program logic may advantageously be implemented as one or more components. The components may advantageously be configured to execute on one or more processors. The components include, but are not limited to, software or hardware components, modules such as software modules, object-oriented software components, class components and task components, processes methods, functions, attributes, procedures, subroutines, segments of program code, drivers, firmware, microcode, circuitry, data, databases, data structures, tables, arrays, and variables.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

There may be many other ways to implement the subject technology. Various functions and elements described herein may be partitioned differently from those shown without departing from the scope of the subject technology. Various modifications to these configurations will be readily apparent to those skilled in the art, and generic principles defined herein may be applied to other configurations. Thus, many changes and modifications may be made to the subject technology, by one having ordinary skill in the art, without departing from the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such “an embodiment” may refer to one or more embodiments and vice versa. A phrase such as “a configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as “a configuration” may refer to one or more configurations and vice versa.

As used herein, the phrase “at least one of” preceding a series of items, with the terms “and” or “or” to separate any of the items, modifies the list as a whole, rather than each member of the list (i.e., each item). The phrase “at least one of” does not require selection of at least one item; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Terms such as “top,” “bottom,” “front,” “rear” and the like as used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Furthermore, to the extent that the term “include,” “have,” or the like is used in the description or the claims, such term is intended to be inclusive in a manner similar to the term “comprise” as “comprise” is interpreted when employed as a transitional word in a claim.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

1-14. (canceled)

15. A system comprising:
a first endovascular device for placement in a first cerebral blood vessel of a patient and comprising a plurality of first electrodes;
a second endovascular device for placement in a second cerebral blood vessel of the patient and comprising a plurality of second electrodes; and
a controller configured to control an electrical generator to pass electrical currents both in a first direction from the first endovascular device to the second endovascular device and in a second direction from the second endovascular device to the first endovascular device along a plurality of pathways that intersect each other to establish a network of intersecting conduction pathways between the first electrodes and the second electrodes during a single treatment period.

16. The system of claim 15, wherein the controller is configured to select a plurality of combinations of electrodes from the first electrodes and the second electrodes to form a plurality of electrode pairs, and wherein to control the electrical generator, the controller is configured to control the electrical generator to pass electrical currents via the plurality of electrode pairs.

17. The system of claim 16, wherein to select the plurality of combinations of electrodes from the first electrodes and second electrodes to form the plurality of electrode pairs, the controller is configured to randomly select the plurality of combinations of electrodes from the first electrodes and second electrodes to form the plurality of electrode pairs.

18. The system of claim 16, wherein to select the plurality of combinations of electrodes from the first electrodes and second electrodes to form the plurality of electrode pairs, the controller is configured to select, according to a preset pattern, the plurality of combinations of electrodes from the first electrodes and second electrodes to form the plurality of electrode pairs.

19. The system of claim 15, wherein the plurality of pathways comprises pathways between a plurality of electrode pairs, each of the electrode pairs of the plurality of electrode pairs including electrodes from each of the plurality of first electrodes and the plurality of second electrodes.

20. The system of claim 19, wherein the plurality of electrode pairs include:
a first pair including a distal-most electrode of the plurality of second electrodes and a proximal-most electrode of the plurality of first electrodes, wherein to pass electrical currents along the plurality of pathways, the controller is configured to control the electrical generator to pass electrical currents along a first pathway between electrodes of the first pair, the first pathway traversing both longitudinally and laterally through brain tissue from the second endovascular device to the first endovascular device; and
a second pair including electrodes that are longitudinally closer to each other than the distal-most electrode of the plurality of second electrodes and the proximal-most electrode of the plurality of first electrodes, wherein to pass electrical currents along the plurality of pathways, the controller is configured to control the electrical generator to pass electrical currents along a second pathway between the electrodes of the second pair.

21. The system of claim 15, wherein the plurality of first electrodes comprises X number of electrodes, the plurality of second electrodes comprises Y number of electrodes, and a number of available pathways between the plurality of first electrodes and the plurality of second electrodes is 2XY, and wherein to pass electrical currents along the plurality of pathways, the controller is configured to control the electrical generator to pass electrical currents along more than half of the 2XY available pathways during the single treatment period.

22. The system of claim 21, wherein X and Y are each 5 to 10.

23. The system of claim 15, wherein to pass electrical currents, the controller is configured to pass electrical currents in the first direction between at least an electrode pair that includes one electrode from the plurality of first electrodes and one electrode from the plurality of second electrodes, and wherein the controller is configured to control the electrical generator to switch, during the single treatment period, a polarity of the electrode pair such that currents pass in the second direction from the second endovascular device to the first endovascular device.

24. The system of claim 15, wherein to pass electrical currents both in the first direction from the first endovascular device to the second endovascular device and in the second direction from the second endovascular device to the first endovascular device, the controller is configured to:
control the electrical generator to deliver electrical stimulation via at least one first electrode of the plurality of first electrodes; and
after delivering the electrical stimulation, control the electrical generator to switch a polarity of the at least one first electrode and use the at least one first electrode as a ground electrode to deliver electrical stimulation.

25. A system comprising:
a first endovascular device for placement in a first cerebral blood vessel of a patient comprising a plurality of first electrodes including X number of electrodes;
a second endovascular device for placement in a second cerebral blood vessel of the patient and comprising a plurality of second electrodes including Y number of electrodes, wherein a number of available pathways of a plurality of pathways between the plurality of first electrodes and the plurality of second electrodes is 2XY; and
a controller configured to control an electrical generator to pass electrical currents along more than half of the 2XY available pathways between the first electrodes and the second electrodes during a single treatment period.

26. The system of claim 25, wherein the controller is configured to select a plurality of combinations of electrodes from the first electrodes and the second electrodes to form a plurality of electrode pairs, and wherein to control the electrical generator, the controller is configured to control the electrical generator to pass electrical currents via the plurality of electrode pairs.

27. The system of claim 26, wherein to select the plurality of combinations of electrodes from the first electrodes and second electrodes to form the plurality of electrode pairs, the controller is configured to randomly select the plurality of combinations of electrodes from the first electrodes and second electrodes to form the plurality of electrode pairs.

28. The system of claim 26, wherein to select the plurality of combinations of electrodes from the first electrodes and second electrodes to form the plurality of electrode pairs, the controller is configured to select, according to a preset pattern, the plurality of combinations of electrodes from the first electrodes and second electrodes to form the plurality of electrode pairs.

29. The system of claim 25, wherein the plurality of pathways comprises pathways between a plurality of electrode pairs, each of the electrode pairs of the plurality of electrode pairs including electrodes from each of the plurality of first electrodes and the plurality of second electrodes.

30. The system of claim 25, wherein X and Y are each 5 to 10.

31. The system of claim 25, wherein to pass electrical currents between the first electrodes and the second electrodes during the single treatment period, the controller is configured to control the electrical generator to pass electrical currents along a plurality of pathways that intersect each other to establish a network of intersecting conduction pathways between the first electrodes and the second electrodes during the single treatment period.

32. The system of claim 25, wherein to pass electrical currents between the first electrodes and the second electrodes during the single treatment period, the controller is configured to control the electrical generator to pass electrical currents both in a first direction from the first endovascular device to the second endovascular device and in a second direction from the second endovascular device to the first endovascular device.

33. The system of claim 32, wherein to pass electrical currents, the controller is configured to pass electrical currents in the first direction between at least an electrode pair that includes one electrode from the plurality of first electrodes and one electrode from the plurality of second electrodes, and wherein the controller is configured to control the electrical generator to switch, during the single treatment period, a polarity of the electrode pair such that currents pass in the second direction from the second endovascular device to the first endovascular device.

34. The system of claim 32, wherein to pass electrical currents both in the first direction from the first endovascular device to the second endovascular device and in the second direction from the second endovascular device to the first endovascular device, the controller is configured to:
control the electrical generator to deliver electrical stimulation via at least one first electrode of the plurality of first electrodes; and
after delivering the electrical stimulation, control the electrical generator to switch a polarity of the at least one first electrode and use the at least one first electrode as a ground electrode to deliver electrical stimulation.
